(19) United States
(12) Patent Application Publication
(10) Pub. No.: US 2023/0056846 A1
(43) Pub. Date: Feb. 23, 2023

(54) METHODS AND COMPOSITIONS FOR CANCER IMMUNOTHERAPY

(71) Applicant: MEMORIAL SLOAN KETTERING CANCER CENTER, New York, NY (US)

(72) Inventors: Luis Alberto Diaz, New York, NY (US); Benoit Rousseau, New York, NY (US); Neil H. Segal, New York, NY (US)

(21) Appl. No.: 17/792,557

(22) PCT Filed: Jan. 13, 2021
(86) PCT No.: PCT/US2021/013212

§ 371 Date: Jul. 13, 2022

Related U.S. Application Data
(60) Provisional application No. 62/960,464, filed on Jan. 13, 2020.

(51) Int. Cl. A61K 31/495; A61K 33/243; C07K 16/28; A61K 9/00; A61P 35/00

(52) U.S. Cl. CPC A61K 31/495; A61K 9/0019; A61K 9/0053; A61K 33/243; A61P 35/00; C07K 16/28

(57) ABSTRACT

In some embodiments the present invention provides methods useful for the treatment of MMR-proficient and/or microsatellite stable (MSS) cancers and also useful for enhancing the immunogenicity of MMR-proficient and/or microsatellite stable (MSS) cancer cells, enhancing the sensitivity of MMR-proficient and/or microsatellite stable (MSS) cancer cells to immune checkpoint blockade, inducing an MMR-deficient mutational signature in MMR-proficient and/or microsatellite stable (MSS) cancer cells, and/or increasing the frequency of both missense and InDel mutations in MMR-proficient and/or microsatellite stable (MSS) cancer cells. In some embodiments such methods involve administration of a combination of temozolomide and cisplatin, or a combination of temozolomide, cisplatin and an immune checkpoint inhibitor, to a subject in need thereof.

### CROSS REFERENCE TO RELATED APPLICATIONS

This application claims the benefit of priority of U.S. Provisional Pat. Application No. 62/960,464 filed on Jan. 13, 2020, the content of which is hereby incorporated by reference in its entirety.

### STATEMENT REGARDING FEDERALLY SPONSORED RESEARCH

This invention was made with government support under CA009512 and CA008748 awarded by the National Institutes of Health. The government has certain rights in the invention.

### INCORPORATION BY REFERENCE

For the purposes of only those jurisdictions that permit incorporation by reference, the references cited in this disclosure are hereby incorporated by reference in their entireties. In addition, any manufacturers’ instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

Worldwide, colorectal cancer (CRC) is the third most common form of cancer in men, with 663,000 cases (10% of the total) and the second most common form of cancer in women, with 571,000 cases (9.4% of the total) per year. Each year there are about 608,000 deaths from colon cancer which is approximately 8% of all cancer deaths making colorectal cancer the fourth most common cause of cancer death (Ferlay, J., et al., “Estimates of worldwide burden of cancer in 2008;” GLOBOCAN 2008. IntJ Cancer, 2010. 127(12): p. 2893-917). In 2012 in the U.S. an estimated 103,170 new cases were diagnosed with 51,690 deaths (American Cancer Society. Cancer Facts and Figures 2011). Treatment of CRC depends largely on the stage of the disease, which is most-commonly rated according to tumor, nodes, and metastasis (TNM) criteria. The initial treatment is surgery. However, post-surgery metastatic disease occurs in 40%-60% of patients and the prognosis for patients who develop advanced metastatic disease is poor. Over the past decade, some progress has been made with systemic therapy for the palliation of advanced colorectal cancer. With the introduction of oxaliplatin, irinotecan, anti-VEGF therapies, and anti-EGFR therapies, the median life expectancy of patients has been increased to about 29 months (Meyerhardt, J.A. and R.J. Mayer, Systemic therapy for colorectal cancer. N Engl J Med, 2005. 352(5): p. 476-87.). Despite these therapeutic advances, patients with unresectable, metastatic and/or recurrent CRC, remain incurable. There is a substantial unmet medical need for more effective and less toxic therapies, especially for those patients with advanced disease that have not responded to, or have become resistant to, the existing standard treatments. The development of novel approaches to treatment is greatly needed in order to improve outcomes in such patients. The present invention addresses such needs, providing new methods of treatment of colorectal cancer and other cancers.

### SUMMARY OF THE INVENTION

Colorectal cancer patients are generally not considered to be candidates for treatment with immune checkpoint inhibitors. However, recently it has been discovered that a subset of colorectal cancer patients exhibiting high levels of microsatellite instability (MSI-H) resulting from a deficiency in DNA mismatch repair (MMR-deficient) are surprisingly susceptible to treatment with immune checkpoint inhibitors. (See Overman et al., “Durable Clinical Benefit with Nivolumab Plus Ipilimumab in DNA Mismatch Repair-Deficient/Microsatellite Instability-High Metastatic Colorectal Cancer; ” J Clin Oncol, 2018: p. JCO2017769901; Overman et al., “Nivolumab in patients with metastatic DNA mismatch repair-deficient or microsatellite instability-high colorectal cancer (CheckMate 142): an open-label, multicentre, phase 2 study; ’’ Lancet Oncol, 2017. 18(9): p. 1182-1191; Le et al., “PD-1 Blockade in Tumors with Mismatch-Repair Deficiency; ” N. Engl. J. Med., 2015. 372(26): p. 2509-20. See also, U.S. Pat. Application Publication US20190023787A1 relating to treatment of MSI-H, MMR-deficient colorectal cancers). But, unlike the case in these MSI-H / MMR-deficient colorectal cancers (which harbor approximately 1,782 mutations per tumor), immune checkpoint blockade remains ineffective in microsatellite stable (MSS) / MMR-proficient colorectal cancers - which harbor substantially fewer mutations per tumor (approximately 73) (Le et al., “PD-1 Blockade in Tumors with Mismatch-Repair Deficiency; ” N. Engl. J. Med., 2015. 372(26): p. 2509-20).

We hypothesized that it might be possible to overcome the resistance to immune checkpoint blockade in microsatellite stable (MSS) / MMR-proficient colorectal cancers by increasing the number of immunogenic mutations and inducing specific mutational profiles to make these cancers resemble MSI-H / MMR-deficient colorectal cancers - thereby possibly rendering these microsatellite stable (MSS) / MMR-proficient colorectal cancers more sensitive to immune checkpoint blockade. In experiments described in the Examples section of this patent disclosure, we tested this hypothesis and found that a combination of two chemotherapeutic agents — temozolomide (TMZ) and the platinum-containing agent cisplatin — had such effects. Importantly, and surprisingly, we found that while colon cancer cells treated with TMZ alone or cisplatin alone remained microsatellite stable (MSS) / MMR-proficient, colon cancer cells treated with the combination of both of these agents were converted to a MSI-H / MMR-deficient phenotype. We also found that the specific mutational profiles induced by the combination of TMZ and cisplatin were completely unlike the mutational profiles induced by treatment with either TMZ alone or cisplatin alone - suggesting that the combination of agents was acting in a synergistic manner to elicit a mutational profile that was qualitatively different to that elicited by the individual agents. We also found that treatment with a combination of TMZ and cisplatin resulted in increased immunogenicity, increased immune-related cytotoxicity, and high levels of tumor immuno-rejection in vivo in preclinical colon cancer models - again in a synergistic manner. Furthermore, we then expanded our studies to include additional cancer types and found that similar effects were also achieved in vivo in preclinical pancreatic cancer models and melanoma models. Building on these discoveries, the present invention provides a variety of new and improved methods and compositions useful in the treatment of cancer.

Accordingly, the present invention provides various methods for treating cancer in subjects in need thereof. In some embodiments such methods involve administering effective amounts of (a) an imidazotetrazine chemotherapeutic agent and (b) a platinum-containing chemotherapeutic agent to a subject with cancer. And in other embodiments such methods involve administering effective amounts of: (a) an imidazotetrazine chemotherapeutic agent, (b) a platinum-containing chemotherapeutic agent, and (c) an immune checkpoint inhibitor, to a subject with cancer. In some embodiments, prior to commencing such treatment the subject is tested to determine if he or she has a MMR-proficient cancer. In some embodiments, prior to commencing such treatment the subject is tested to determine if he or she has a microsatellite stable (MSS) cancer.

In other embodiments, the present invention provides methods of enhancing the immunogenicity of cancer cells (such as MMR-proficient and/or microsatellite stable (MSS) cancer cells), such methods comprising contacting the cancer cells with an effective amount of an imidazotetrazine chemotherapeutic agent and a platinum-containing chemotherapeutic agent. In some such embodiments the cancer cells are in a subject.

In yet other embodiments, the present invention provides methods of enhancing the sensitivity cancer cells to immune checkpoint blockade, such methods comprising contacting the cancer cells with an effective amount of an imidazotetrazine chemotherapeutic agent and a platinum-containing chemotherapeutic agent. In some such embodiments the cancer cells are in a subject.

In further embodiments, the present invention provides methods of inducing an MMR-deficient mutational signature in MMR-proficient and/or microsatellite stable (MSS) cancer cells, such methods comprising contacting the cancer cells with an effective amount of temozolomide and a platinum-containing chemotherapeutic agent. In some such embodiments the cancer cells are in a subject.

In still further embodiments, the present invention provides methods of increasing the frequency of both missense and InDel mutations in MMR-proficient and/or microsatellite stable (MSS) cancer cells, such methods comprising contacting the cancer cells with an effective amount of an imidazotetrazine chemotherapeutic agent and a platinum-containing chemotherapeutic agent. In some such embodiments the cancer cells are in a subject.

Each of the methods described above or elsewhere herein can be employed in the treatment of cancers in a variety of subjects. For example, in some embodiments the methods described above or elsewhere herein are used to treat subjects that have a MMR-proficient cancer. Similarly, in some embodiments the methods described above or elsewhere herein are used to treat subjects that have a microsatellite stable (MSS) cancer. In some embodiments the methods described above or elsewhere herein are used to treat subjects that have colorectal cancer. In some embodiments the methods described above or elsewhere herein are used to treat subjects that have pancreatic cancer. In some embodiments the methods described above or elsewhere herein are used to treat subjects that have melanoma. In some embodiments the subjects have previously had surgery to remove a tumor (e.g., a colorectal tumor). In some embodiments the subjects have a cancer (e.g., a colorectal cancer) that is not resectable. In some embodiments the subjects have locally advanced cancer. In some embodiments the subjects have metastatic cancer. In some embodiments the subjects have a cancer (e.g., a colorectal cancer) that is resistant to one or more immune checkpoint inhibitors. In some embodiments the subjects have a cancer (e.g., a colorectal cancer) that is resistant to nivolumab. In some embodiments the subjects have a cancer (e.g., a colorectal cancer) that is resistant to one or more chemotherapeutic agents.

Similarly, each of the methods described above or elsewhere herein for affecting biological properties of cancer cells (e.g., to increase their immunogenicity, or to increase their sensitivity to immune checkpoint blockade, or to induce missense mutations, or to induce InDel mutations, and/or to induce a mutational signature of the type associated with MMR-deficiency) can be used on a variety of cell types. In some such embodiments the methods described above or elsewhere herein can be used on MMR-proficient cancer cells. In some embodiments the methods described above or elsewhere herein can be used on microsatellite stable (MSS) cancer cells. In some embodiments the methods described above or elsewhere herein can be used on colorectal cancer cells. In some embodiments the methods described above or elsewhere herein can be used on pancreatic cancer cells. In some embodiments the methods described above or elsewhere herein can be used on melanoma cells.

Many of the methods described above or elsewhere herein involve the use of various active agents or combinations of active agents. For example, many of the embodiments of the present invention involve the use of imidazotetrazine chemotherapeutic agents. In some such embodiments the imidazotetrazine chemotherapeutic agent is selected from the group consisting of temozolomide (TMZ) and dacarbazine. In some such embodiments the imidazotetrazine chemotherapeutic agent is TMZ.

Similarly, many of the embodiments of the present invention involve the use of platinum-containing chemotherapeutic agents. In some such embodiments the platinum-containing chemotherapeutic agent is selected from the group consisting of cisplatin, carboplatin and oxaliplatin. In some such embodiments the platinum-containing chemotherapeutic agent is cisplatin.

And many embodiments of the present invention involve the use of immune checkpoint inhibitors. In some such embodiments the immune checkpoint inhibitor is a PD-1, PD-L1, PD-L2 or CTLA-4 inhibitor. In some such embodiments the immune checkpoint inhibitor is selected from the group consisting of: nivolumab, pembrolizumab, tremelimumab, ipilimumab, cemiplimab, MPDL3280A, AMP-224, AMP-514 and PDR001, atezolizumab, Avelumab, Durvalumab, BMS-936559, CK-301, tislelizumab, toripalimab, envafolimab, HLX10, and HLX20.

These and other embodiments of the invention are further described in the “Brief Description of the Drawings,” “Detailed Description,” “Examples,” “Drawings,” and “Claims” sections of this patent disclosure, each of which sections is intended to be read in conjunction with, and in the context of, all other sections of the present patent disclosure. Furthermore, one of skill in the art will recognize that the various embodiments of the present invention described herein can be combined in various ways, and that such combinations are within the scope of the present invention.

### BRIEF DESCRIPTION OF THE DRAWINGS

FIGS. 1A-E. The combination of TMZ and cisplatin (CDDP) synergizes for cytotoxicity and increases immunogenicity in CT26 colon cancer cells by converting them to an MSI-high phenotype. FIG. 1A. Results of viability assay performed with CT26 cells treated with the agents indicated. FIG. 1B. Results of viability assay performed with CT26 MSH2-/- MMRd cells treated with the agents indicated. Higher activity in the cisplatin-treated cells compared to the parental CT26 cells suggests an involvement of the MMR machinery to prevent cytotoxicity. FIGS. 1C, D. Confluence and cytotoxicity assessed using caspase3/5 fluorescent staining in syngeneic cocultures of activated immune cells isolated from spleen and CT26 parental or CT26 MSH2-/- cells. CT26 cells were treated for 8 weeks by low dose TMZ, CDDP or TMZ/CDDP combination (drugs were removed one week before the experiment). The lefthand graphs in FIG. 1C and FIG. 1 D are data without anti PD-1 treatment; The right-hand graphs in FIG. 1C and FIG. 1 D are data with anti PD-1 treatment (0.25 ug/mL anti PD-1). CT26 parental cells treated for 8W with low dose TMZ/CDDP combination display higher immunogenicity in vitro than the cells treated for 8W with the single agents or vehicle. This effect is amplified by PD-1 blockade. FIG. 1E. Whole exome sequencing at 250X of CT26 cells treated for 8W with vehicle, temozolomide 20 µM, cisplatin 0.5 µM, or a combination of temozolomide 20 µM and Cisplatin 0.5 µM followed by 1 week of wash out. While moderate accumulation of missense mutations and novel frameshift (FS) variants was observed with single agents, the combination of TMZ and CDDP synergized - increasing the tumor mutation burden (TMB) by 125 mt/Mb, increasing the ratio of InDels/FS variants overNSSNV, resulting in an MSI-high genomic phenotype.

FIG. 2. Analyses of mutational signatures showed that chronic exposure to the combination of temozolomide and cisplatin generated a novel MMRd mutational signature for both single base substitutions (SBS) and Indels. The figure shows SBS signatures following 8 weeks of chronic treatment of CT26 cells with either temozolomide 20 µM, cisplatin 0.5 µM, the combination of temozolomide 20 µM and cisplatin 0.5 µM, or vehicle, followed by 1 week of wash out. Cells treated with temozolomide or cisplatin as single agents exhibited very similar SBS signatures (SBS17b, SBS37) consistent with a specific DNA damage process related to the CT26 background. Cells treated with the combination of both temozolomide and cisplatin acquired an MMRd-like mutational signature signature. The combination of temozolomide and cisplatin together appeared to synergize for the accumulation of a predominantly MMRd SBS mutational profile not seen when the agents were used separately, with 70% of the novel mutations being within the MMRd signature spectrum. Indel signature analyses (data not in figure) also showed that cells treated with vehicle alone acquired mainly long deletions within repeated sequences, while cells treated with temozolomide, cisplatin, or the combination of TMZ and cisplatin, acquired predominantly short deletions within long poly T sequences. The cells treated with the combination of temozolomide and cisplatin presented an Indel profile typical of MMR-deficiency, with most of the deletions in poly T regions, defining microsatellites. The Indel profiles of cells treated with the single agents was scattered due to the low number of Indels observed in these conditions and did not generate InDel profiles compatible with MMRd.

FIGS. 3A-C. Treatment of CT26 colon cancer cells with the combination of TMZ and cisplatin induces an inflamed immune state resulting in delayed tumor growth. FIG. 3A. Syngeneic BALB/C mice were implanted subcutaneously with CT26 cells pretreated for 8 weeks with vehicle alone or the combination of temozolomide 20 µM and Cisplatin 0.5 µM (referred to as “TC1” in the figure), followed by 1 week of wash out. When the combination of temozolomide and cisplatin was used, some tumors were spontaneously rejected (2/6). Two different amounts cells were injected - either 1 million cells (1M) or 0.5 million cells (0.5M) — as shown. The tumor growth rate was faster when 1M cells were injected than when 0.5M cells were injected. FIG. 3B. Tumors from the different treatment conditions were stained by immunohistochemistry using antibodies specific for Ki67, CD3, CD4, CD8, Iba1, and PD-L1. Representative micrographs are presented. FIG. 3C. Graphical representation of quantification of immunohistochemical staining is presented. An automated system was used to count stained cells and quantify immunohistochemical staining intensity for Ki67, CD3, CD4, CD8, Iba1, and PD-L1 in 20 randomly-selected 1 mm² fields per sample (N=2 tumors per condition).

FIGS. 4A-B. Treatment of colon cancer cells with TMZ and cisplatin induces a high frameshift load leading to the spontaneous immuno-rejection of tumors in a preclinical mouse model. FIG. 4A. Syngeneic BALB/C mice were implanted subcutaneously with CT26 colon cancer cells pre-treated for 8 weeks with vehicle alone, temozolomide (TMZ) 20 µM, cisplatin (CDDP) 0.5 µM, or a combination of temozolomide 20 µM and cisplatin 0.5 µM (“Combo”) followed by 1W of wash out (N=10 per treatment group) and then randomly assigned to either an IgG isotype control group or anti PD-1 treatment group (6 mg/kg anti PD-1 twice weekly started). Treatment with either anti PD-1 or the IgG isotype control was commenced when the tumors reached a volume of 200 mm³. While no significant difference in tumor growth compared to control was observed in the groups treated with temozolomide alone or cisplatin alone (despite an increase in tumor mutational burden), tumors generated from cells treated with the combination of both temozolomide and cisplatin displayed spontaneous immuno-rejection without need to initiate anti-PD-1 treatment in 9 out of 10 tumors. The MSH2 KO cell line was used as a positive control for PD-1 blockade. FIG. 4B. Tumors were subjected to whole exome sequencing at 250x and immunoediting was studied by comparing mutation losses before and after engraftment. A specific 20-fold increase in mutation losses was observed for frameshift mutations in cells treated with the combination of TMZ and CDDP as compared to cells treated with the single agents - in line with active immunoediting of this immunogenic mutation class.

FIG. 5. Transition and transversion mutational profiles in CT26 colon cancer cells treated for 8 weeks with temozolomide (TMZ) 20 µM, cisplatin (CDDP) 0.5 µM or a combination of temozolomide 20 µM and cisplatin 0.5 µM (TMZ + CDDP) followed by 1 week of wash out. The data shows that the combination of temozolomide and cisplatin leads to a unique T to C transition rich profile typical of the MMRd mutational signature SBS21 - which such transitions are not enriched when either temozolomide or cisplatin are used as single agents.

FIG. 6. Syngeneic C57BL/6 mice were implanted subcutaneously with Pan02 pancreatic cancer cells pre-treated for 8 weeks with either vehicle alone (upper left graph), temozolomide 20 µM (TMZ, upper right graph), cisplatin 0.5 µM (CDDP, lower left graph) or a combination of both temozolomide 20 µM and cisplatin 0.5 µM (Combo, lower right graph) followed by 1 week of wash out (N=6 mice per treatment group). Each line on each of the graphs represents results from a different mouse. Tumors generated from pancreatic cancer cells treated with the combination of both temozolomide and cisplatin exhibited increased spontaneous tumor rejection and delayed growth as compared to tumors generated from pancreatic cancer cells treated with the single agents.

FIG. 7. Syngeneic C57BL/6 mice were implanted subcutaneously with 1 million B16/F10 melanoma cells pre-treated for 8 weeks with either vehicle alone (B16/F10) or a combination of both temozolomide 20 µM and cisplatin 0.5 µM (TMZ+CDDP) followed by 1 week of wash out (N=5 mice per condition). The tumor volume data shown is averaged across mice. While all tumors grew in the vehicle group condition, all tumors were spontaneously rejected in the TMZ+CDDP treatment group.

### DETAILED DESCRIPTION

Some of the embodiments of the present invention are described in the “Summary of the Invention,” “Examples,” “Brief Description of the Drawings,” and “Drawings” sections of this patent disclosure. This Detailed Description section provides certain additional embodiments and certain additional description and details relating to embodiments described elsewhere herein and is intended to be read in conjunction with all other sections of the present patent disclosure.

### Definitions and Abbreviations

In order that the present invention can be more readily understood, certain terms are defined herein. Additional definitions are set forth throughout the disclosure. Unless defined otherwise, all technical and scientific terms used herein have the same meaning as commonly understood by one of ordinary skill in the art to which this invention is related

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values ±10% of the stated number.

Numbers in parentheses or superscript following text in this patent disclosure refer to the numbered references provided in the “Reference List” section at the end of this patent disclosure.

Wherever embodiments are described with the language “comprising,” otherwise analogous embodiments described in terms of “consisting of” and/or “consisting essentially of” are included.

As used herein, the term “antibody” encompasses polyclonal antibodies; monoclonal antibodies; multi-specific antibodies, such as bispecific antibodies generated from at least two intact antibodies; humanized antibodies; human antibodies; chimeric antibodies; fusion proteins comprising an antigen-determination portion of an antibody; and any other modified immunoglobulin molecule comprising an antigen recognition site, so long as the antibodies exhibit the desired biological activity.

As used herein the terms “inhibiting” and “blocking” are used interchangeably, as are the terms “inhibit” or “block” and the terms “inhibitor” or “blocker.” The terms “inhibit” and “block” refer to any detectable and statistically significant decrease in a given biological activity.

As used herein a “subject” is any individual for whom diagnosis, prognosis, or therapy is desired. In some embodiments the subjects are mammalian subjects, including humans, domestic animals, farm animals, sports animals, and zoo animals. In some embodiments the subjects are non-human primates. In some embodiments the subjects are murine subjects. In some embodiments the subjects are humans.

As used herein the abbreviation “CDDP” refers to cisplatin.

As used herein the abbreviation “CNV” refers to copy number variant.

As used herein the abbreviation “CRC” refers to colorectal cancer.

As used herein the abbreviation CTLA-4 refers to cytotoxic T-lymphocyte-associated protein 4.

As used herein the abbreviation “FS” refers to frame shift mutations.

As used herein the abbreviation “Iba1” refers to ionized calcium binding adaptor molecule 1.

As used herein the abbreviation “ICI” refers to immune checkpoint inhibition or immune checkpoint inhibitors.

As used herein the abbreviation “ICB” refers to immune checkpoint blockers immune checkpoint blockade.

As used herein the abbreviation “InDel” refers to insertion and deletion mutations.

As used herein the abbreviation “IV” refers to intravenous.

As used herein the abbreviation “MMR” refers to DNA mismatch repair.

As used herein the abbreviations “MMRd” and “MMR-deficient” refer to DNA mismatch repair deficient.

As used herein the abbreviations “MMRp” and “MMR-proficient” refer to DNA mismatch repair proficient.

As used herein the abbreviation “MSI” refers to microsatellite instability.

As used herein the abbreviation MSI-H refers to high levels of microsatellite instability.

As used herein the abbreviation “MSS” refers to microsatellite stability.

As used herein the abbreviation “NSSNV” refers to non-synonymous single nucleotide variants.

As used herein the abbreviation “PD-1” refers to Programmed Death 1, which is also known as Programmed Death Protein 1 or Programmed Cell Death Protein 1.

As used herein the abbreviation PD-L1 refers to Programmed Cell Death Ligand 1 -which is a ligand for PD-1.

As used herein the abbreviation PD-L2 refers to Programmed Cell Death Ligand 2.

As used herein the abbreviation “SBS” refers to single base substitution.

As used herein the abbreviation “TMB” refers to tumor mutational burden.

As used herein the abbreviation “TMZ” refers to temozolomide.

Other abbreviations and definitions may be provided elsewhere in this patent specification, or may be well known in the art.

### Active Agents

Several embodiments of the present invention involve the use of various active agents or combinations of agents, including, but not limited to, imidazotetrazines (such as temozolomide), platinum-containing chemotherapeutic agents (such as cisplatin, carboplatin or oxaliplatin), and various immune checkpoint inhibitors.

In some embodiments immune checkpoint inhibitors are used in the compositions and methods of the present invention. Immune checkpoint inhibitors that can be used in accordance with the present invention include PD-1, PD-L1, PD-L2 and CTLA-4 inhibitors. In some embodiments a PD-1 inhibitor is used. In some embodiments a PD-L1 inhibitor is used. In some embodiments a PD-L2 inhibitor is used. In some embodiments a CTLA-4 inhibitor is used. In some embodiments the PD-1, PD-L1, PD-L2 and/or CTLA-4 inhibitors are, or comprise, an antibodies or antigen-binding fragments thereof.

In some embodiments an immune checkpoint inhibitor is selected from the group consisting of nivolumab, pembrolizumab, tremelimumab, ipilimumab. cemiplimab, MPDL3280A, AMP-224, AMP-514 and PDR001, atezolizumab, Avelumab, Durvalumab, BMS-936559 and CK-301.

In some embodiments a PD-1 inhibitor is selected from the group consisting of pembrolizumab, nivolumab, cemiplimab, AMP-224, AMP-514, and PDR001. In some embodiments the immune checkpoint inhibitor is nivolumab.

In some embodiments a PD-L1 inhibitor is selected from the group consisting of atezolizumab, avelumab, durvalumab, BMS-936559, CK-301. In some embodiments the immune checkpoint inhibitor is or comprises an anti- PD-L1 antibody or an antigen-binding fragment thereof.

In some embodiments a CTLA-4 inhibitor is selected from the group consisting of ipilimumab or tremelimumab. In some embodiments the immune checkpoint inhibitor is or comprises an anti-CTLA4 antibody or an antigen-binding fragment thereof.

In some embodiments imidazotetrazines are used in the compositions and methods of the present invention. Imidazotetrazines are a class of bicyclic aromatic heterocycles that include the DNA temozolomide (TMZ) and dacarbazine. In some embodiments the imidazotetrazine is selected from the group consisting of TMZ and dacarbazine. In some embodiments the imidazotetrazine is TMZ. Temozolomide (TMZ) is an alkylating agent that can cause tumors to accumulate mutations. TMZ leads to incorporation of 06-meG•C and 06-meG•T mutations, which are recognized by a MMR pathway and lead to a ‘futile repair cycle’ and accumulation of G·C ➔ A·T mutations. TMZ is typically administered orally. TMZ alone has been evaluated in CRC and showed modest activity in recent phase 2 studies of TMZ monotherapy, in pretreated metastatic colorectal cancer with MGMT promoter methylation (Pietrantonio et al., “Activity of temozolomide in patients with advanced chemorefractory colorectal cancer and MGMT promoter methylation; ” Annals of Oncology, 2013. 25(2): p. 404-408.; Amatu et al., “Tumor MGMT promoter hypermethylation changes over time limit temozolomide efficacy in a phase II trial for metastatic colorectal cancer; ” Ann Oncol, 2016. 27(6): p. 1062-7; Calegari et al., “A phase 2 study of temozolomide in pretreated metastatic colorectal cancer with MGMTpromoter methylation.” Br J Cancer, 2017. 116(10): p. 1279-1286; Hochhauser et al., “A phase II study of temozolomide in patients with advanced aerodigestive tract and colorectal cancers and methylation of the 06-methylguanine-DNA methyltransferase promoter; ” Mol Cancer Ther, 2013. 12(5): p. 809-18).

In some embodiments, platinum-containing chemotherapeutic agents are used in the compositions and methods of the present invention. The platinum-containing chemotherapeutic agents cisplatin, carboplatin, and/or oxaliplatin can be used in accordance with the present invention. These agents are typically administered intravenously. These agents are alkylating agents and interfere with DNA replication mostly by creating drug-DNA adducts crosslinking two adjacent guanines (GpG, 65%) or an adenine and a guanine (5′-ApG-3′, 25%). (Boot et al., “In-depth characterization of the cisplatin mutational signature in human cell lines and in esophageal and liver tumors;” Genome Res, 2018. 28(5): p. 654-665).

In some embodiments, the methods of the present invention can be carried out using analogues, homologues, variants, or derivatives that are equivalents of any of the specific active agents described herein. Such analogues, homologues, variants, or derivatives should retain the key functional properties of the specific molecules described herein. For example, in the case of PD-1 and/or PD-L 1 inhibitors, any suitable analogue, homologue, variant, or derivative of such an agent can be used provided that it retains PD-1 and/or PD-L1 inhibitory activity. Similarly, in the case of temozolomide, any suitable analogue, homologue, variant, or derivative of temozolomide can be used provided that it retains mutation-inducing activity comparable to that of temozolomide. Likewise, in the case of the platinum-containing chemotherapeutic agents, cisplatin, carboplatin and/or oxaliplatin, any suitable analogue, homologue, variant, or derivative of these agents can be used provided that it retains mutation-inducing activity comparable to that of cisplatin.

### Pharmaceutical Compositions

In certain embodiments, the present invention provides pharmaceutical compositions comprising at least one active agent as described herein. The term “pharmaceutical composition” refers to a preparation that is in such form as to permit the biological activity of the active agent or agents (e.g imidazotetrazines, platinum-containing chemotherapeutic agents, and/or various immune checkpoint inhibitors) to be effective and which contains no additional components that are unacceptably toxic to a subject to which the composition may be administered. Such compositions can be sterile. Typically, such compositions comprise a pharmaceutically acceptable carrier. Examples of pharmaceutically acceptable carriers include, but are not limited to, physiological saline. In some embodiments pharmaceutical compositions can comprise one or more of: a buffer (e.g., acetate, phosphate, or citrate buffer), a surfactant (e.g., polysorbate), a stabilizing agent (e.g., human albumin), a preservative (e.g., benzyl alcohol), an absorption promoter to enhance bioavailability and/or other conventional solubilizing or dispersing agents.

In some embodiments the present invention provides pharmaceutical compositions comprising: (a) an imidazotetrazine chemotherapeutic agent and (b) a platinum-containing chemotherapeutic agent. A pharmaceutical composition comprising: (a) TMZ and (b) cisplatin. In some embodiments the present invention provides pharmaceutical compositions comprising: (a) a platinum-containing chemotherapeutic agent, and (b) an immune checkpoint inhibitor. In some embodiments the present invention provides pharmaceutical compositions comprising: (a) cisplatin, and (b) nivolumab. In some embodiments the present invention provides pharmaceutical compositions comprising: (a) an imidazotetrazine chemotherapeutic agent, (b) a platinum-containing chemotherapeutic agent, and (c) an immune checkpoint inhibitor. In some embodiments the present invention provides pharmaceutical compositions comprising: (a) temozolomide, (b) cisplatin, and (c) nivolumab. In some embodiments such pharmaceutical compositions are used in the treatment of a MMR-proficient and/or microsatellite stable cancer in a subject in need thereof. In some embodiments such pharmaceutical compositions are used in treatment of a MMR-proficient and/or microsatellite stable colorectal cancer in a subject in need thereof. In some embodiments such pharmaceutical compositions are used in treatment of a MMR-proficient and/or microsatellite stable pancreatic cancer in a subject in need thereof. In some embodiments such pharmaceutical compositions are used in treatment of a MMR-proficient and/or microsatellite stable melanoma in a subject in need thereof. In some embodiments such pharmaceutical compositions are used.

### Methods of Treatment

The present invention provides various methods of treatment. For example, in some embodiments the present invention provides treatment methods that comprise administering effective amounts of one or more of the active agents described herein to subjects in need thereof.

As used herein, the terms “treat,” “treating,” and “treatment” refer achieving, and/or administering an agent or agents to a subject in order to achieve, to a detectable degree, an improvement in one or more clinical indicators or symptoms of a disease or medical condition or a desired biological outcome in a subject, or in tissues or cells in a subject. For example, such terms include, but are not limited to, reducing the rate of growth of a tumor (or of cancer cells), halting the growth of a tumor (or of cancer cells), causing regression of a tumor (or of cancer cells), reducing the size of a tumor (for example as measured in terms of tumor volume or tumor mass), reducing the grade of a tumor, eliminating a tumor (or tumor cells), preventing, delaying, or slowing recurrence (rebound) of a cancer/tumor, improving symptoms associated with a cancer/tumor, improving survival from a cancer/tumor, inhibiting or reducing spreading of a cancer/tumor (e.g., metastases), and the like. Importantly, in the context of the present invention the terms “treat,” “treating,” and “treatment” also refer to methods that result in one or more of: (a) immune rejection of a tumor or tumor cells, (b) an enhancement of the immunogenicity of cancer cells (such as of MMR-proficient and/or microsatellite stable (MMS) cancer cells), (c) an enhancement of the sensitivity of cancer cells (such as MMR-proficient and/or microsatellite stable (MSS) cancer cells) to immune checkpoint blockade, (d) induction of an MMR-deficient mutational signature in cancer cells (such as MMR-proficient and/or microsatellite stable (MSS) cancer cells), and an increase in the frequency or number of both missense and InDel mutations in cancer cells (such as MMR-proficient and/or microsatellite stable (MSS) cancer cells) - each of which are desirable biological outcomes of the present methods.

As used herein the term “subject” encompasses all mammalian species, including, but not limited to, humans, non-human primates, dogs, cats, rodents (such as rats, mice and guinea pigs), cows, pigs, sheep, goats, horses, and the like - including all mammalian animal species used in animal husbandry, as well as animals kept as pets and in zoos, etc. In preferred embodiments the subjects are human.

In some embodiments the present invention may be directed to treatment of any cancer type in a subject in need thereof. In some embodiments of the subject has a MMR-proficient cancer. In some embodiments the subject has a microsatellite stable (MSS) cancer. In some embodiments the subject has metastatic cancer. In some embodiments subject has locally advanced cancer. In some embodiments the subject has colorectal cancer. In some embodiments the subject has pancreatic cancer. In some embodiments the subject has melanoma.

In some embodiments the present invention may be directed to treatment of a cancer that was previously resistant to treatment with an immune checkpoint inhibitor. As used herein, the terms “resistant” and “resistance” are used consistent with their normal usage in the art and consistent with the understanding of those term by physicians who treat cancer (e.g., oncologists). For example, consistent with its usual meaning in the art, a tumor or a subject may be considered “resistant” to a certain treatment method or treatment with a certain agent (or combination of agents), if, despite using that method or administering that agent (or combination of agents), a subject’s tumor (or tumor cells) grows, and/or progresses, and/or spreads, and/or metastasizes, and/or recurs. In some instances, a tumor may initially be sensitive to treatment with a certain method or agent (or combination of agents), but later became resistant to such treatment.

In some embodiments the subject has a cancer (e.g., colorectal cancer) that has recurred following a prior treatment with other compositions or methods, including, but not limited to, chemotherapy, radiation therapy, or surgical resection, or any combination thereof.

In some embodiments the subject has a cancer that has not previously been treated.

As used herein the term “effective amount” refers to an amount of an active agent as described herein that when administered (alone or in combination with an additional therapeutic/prophylactic agent) to a cell, tissue, or subject, or contacted with a cell or tissue, is effective to achieve, to a detectable degree, one or more of the desirable biological outcomes or clinical improvements described above in the context of the “treatment” definition or described elsewhere herein. When applied to a “combination” of agents, an effective dose refers to combined amounts of the active agents that result in the desired clinical improvement or biological outcome, whether the combination of agents is administered simultaneously (e.g., delivered in admixture together or delivered simultaneously by different routes or in different dosage forms) or administered sequentially (e.g., delivered by different routes and/or in different dosage forms and/or at different times). The term “combination” encompasses all such administration.

An appropriate “effective” amount in any individual case may be determined using standard techniques known in the art, such as dose escalation studies, and may be determined taking into account such factors as the desired route of administration (e.g., oral vs. intravenous), desired frequency of dosing, etc. Furthermore, an “effective amount” may be determined in the context of any combination administration to be used. One of skill in the art can readily perform such dosing studies (whether using single agents or combinations of agents) to determine appropriate doses to use, for example using assays that involve administration of the agents described herein to subjects - such as animal subjects routinely used in the pharmaceutical sciences for performing dosing studies.

For example, in some embodiments the dose of an active agent of the invention may be calculated based on studies in humans or other mammals carried out to determine efficacy and/or effective amounts of the active agent. The dose may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In those embodiments described herein that refer to specific doses of agents to be administered based on animal studies (e.g., mouse studies), one of skill in the art can readily determine comparable doses for human studies, for example using the types of dosing studies (e.g., dose escalation studies) and calculations known in the art and/or described herein.

In some embodiments one or more of the active agents is used at approximately its maximum tolerated dose, for example as determined in phase I clinical trials and/or in dose escalation studies. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose. In some embodiments one or more of the active agents is used at about 20% of its maximum tolerated dose. In some embodiments one or more of the active agents is used at about 10% of its maximum tolerated dose.

In some embodiments where temozolomide (TMZ) or another imidazotetrazine chemotherapeutic agent is administered, it is administered at a dose of about 50-200 mg/m2/day, or about 100-150 mg/m2/day, or about 150-200 mg/m2/day, or about 200-250 mg/m2/day, or about 75 mg/m2/day, or about 50-250 mg/m2/day, or about 100-200 mg/m2/day. In some such embodiments the TMZ is administered daily, or is administered in a cycle whereby it is administered daily for several days (e.g., for 2, 3, 4, 5, 6, or 7 days), followed by a break of about 1-3 weeks - before repeating the cycle. In some embodiments the temozolomide (TMZ) is administered daily for days 1 to 5 every 4 weeks. The total duration of the TMZ treatment regimen is typically from two months to two years. In some embodiments the total duration of the TMZ treatment regimen is about 1 month, or about 2 months, or about 3 months, or about 4 months, or about 5 months, or about 6 months, or about 7 months, or about 8 months, or about 9 months, or about 10 months, or about 11 months, or about 12 months, or more.

In some embodiments where cisplatin, or another platinum-containing chemotherapeutic agent is administered, it is administered at a dose of about 20 mg/m2, or about 30 mg/m2, or about 40 mg/m2, or about 50 mg/m2 or about 60 mg/m2 or about 70 mg/m2 or about 75 mg/m2 or about 80 mg/m2 or about 90 mg/m2 or about 100 mg/m2 - via IV infusion. In some such embodiments the IV infusion is administered every day, or every week, or every two weeks (Q2W), or every three weeks, or every 4 weeks. In some embodiments cisplatin is administered at a dose of about 40 mg/m2 via IV infusion every two weeks (Q2W). The total duration of the cisplatin treatment regimen is typically from two months to two years. In some embodiments the total duration of the cisplatin treatment regimen is about 1 month, or about 2 months, or about 3 months, or about 4 months, or about 5 months, or about 6 months, or about 7 months, or about 8 months, or about 9 months, or about 10 months, or about 11 months, or about 12 months, or more.

In some embodiments, where an immune checkpoint inhibitory antibody (such as anti-PD-1, PD-L1 or CTLA-4 antibody) is administered, it is administered at a dose ranging from about 0.1 mg/kg to at about 10.0 mg/kg body weight and is administered once about every 1, 2, 3, or 4 weeks. Similarly, in some embodiments, where an immune checkpoint inhibitory antibody (such as anti-PD-1, PD-L1 or CTLA-4 antibody) is administered, it is administered at a dose ranging from about 1 mg/kg to at about 10.0 mg/kg body weight and is administered once about every 1, 2, 3, or 4 weeks. And in some embodiments, where an immune checkpoint inhibitory antibody (such as anti-PD-1, PD-L1 or CTLA-4 antibody) is administered, it is administered at a dose ranging from about 3 mg/kg to at about 10.0 mg/kg body weight and is administered once about every 1, 2, 3 or, 4 weeks.

For example, in some embodiments an immune checkpoint inhibitory antibody (such as anti-PD-1, PD-L1 or CTLA-4 antibody) is administered at a dose of about 3 mg/kg about every 2 weeks. Similarly, in some embodiments an immune checkpoint inhibitory antibody (such as anti-PD-1, PD-L1 or CTLA-4 antibody) is administered at a dose of about 6 mg/kg about every 4 weeks.

In some embodiments, where an immune checkpoint inhibitory antibody (such as anti-PD-1, PD-L1 or CTLA-4 antibody) is administered, it is administered at a dose ranging from about 8 mg total dose to at about 800 mg total dose and is administered once about every 1, 2, 3, or 4 weeks. Similarly, in some embodiments an immune checkpoint inhibitory antibody (such as anti-PD-1, PD-L1 or CTLA-4 antibody) is administered at a dose ranging from about 80 mg total dose to at about 800 mg total dose and is administered once about every 1, 2, 3, or 4 weeks. And in some embodiments an immune checkpoint inhibitory antibody (such as anti-PD-1, PD-L1 or CTLA-4 antibody) is administered at a dose ranging from about 240 mg total dose at about 800 mg total dose and is administered once about every 1, 2, 3 or, 4 weeks. And in some embodiments an immune checkpoint inhibitory antibody (such as anti-PD-1, PD-L1 or CTLA-4 antibody) is administered at a dose ranging from about 240 mg total dose at about 540 mg total dose and is administered once about every 1, 2, 3 or, 4 weeks. Similarly, in some embodiments an immune checkpoint inhibitory antibody (such as anti-PD-1, PD-L1 or CTLA-4 antibody) is administered at a dose ranging from about 240 mg total dose at about 480 mg total dose and is administered once about every 1, 2, 3 or, 4 weeks.

For example, in some embodiments an immune checkpoint inhibitory antibody (such as anti-PD-1, PD-L1 or CTLA-4 antibody) is administered at a dose of about 240 mg about every 2 weeks. Similarly, in some embodiments an immune checkpoint inhibitory antibody (such as anti-PD-1, PD-L1 or CTLA-4 antibody) is administered at a dose of about 480 mg about every 4 weeks.

Additional examples of doses of an immune checkpoint inhibitory antibody (such as anti-PD-1, PD-L1 or CTLA-4 antibody) that can be administered are about 80 mg, about 160 mg, about 240 mg, about 320 mg, about 400 mg, about 480 mg, about 560 mg, about 640 mg, about 720 mg, or about 800 mg - total dose, with such doses administered about every 1, 2, 3 or, 4 weeks.

Typically, in those embodiments, where an immune checkpoint inhibitory antibody (such as anti-PD-1, PD-L1 or CTLA-4 antibody) is administered, it is administered by IV infusion. The time course over which a dose of the immune checkpoint inhibitory antibody is infused may be any suitable time. In some embodiments the immune checkpoint inhibitory antibody is infused over a time period of about 30 minutes. In some embodiments the immune checkpoint inhibitory antibody is infused over a time period of about 60 minutes.

The total duration of the immune checkpoint inhibitor treatment regimen is typically from two months to two years. In some embodiments the total duration of the immune checkpoint inhibitor treatment regimen is about 1 month, or about 2 months, or about 3 months, or about 4 months, or about 5 months, or about 6 months, or about 7 months, or about 8 months, or about 9 months, or about 10 months, or about 11 months, or about 12 months, or more.

In carrying out the treatment methods described herein, any suitable method or route of administration can be used to deliver/administer the active agents or combinations thereof described herein. In some embodiments systemic administration may be employed, for example, oral or intravenous administration, or any other suitable method or route of systemic administration known in the art. In some embodiments intratumoral administration may be employed. For example, the active agents described herein may be administered either systemically or locally by injection, by infusion through a catheter, using an implantable drug delivery device, or by any other means known in the art. One of skill in the art will be able to select the appropriate administration method or route depending on the situation, for example depending on the nature of the active agent (e.g., its bioavailability, stability, half-life, etc.). For example, in some embodiments imidazotetrazine chemotherapeutic agents (such as temozolomide) are administered orally. In some embodiments, imidazotetrazine chemotherapeutic agents (such as temozolomide) administered by IV infusion. In some embodiments, platinum-containing chemotherapeutic agents (such as cisplatin) are administered by IV infusion. In some embodiments, platinum-containing chemotherapeuticagents (such as cisplatin) are administered by IV infusion.

In some embodiments, combinations of the various active agents described herein are administered sequentially. In some embodiments, combinations the various active agents described herein are administered simultaneously. For example, combinations of the active agents described herein can be administered simultaneously (e.g., administered in admixture together or administered by different routes or in different dosage forms) or sequentially (e.g., administered by different routes and/or in different dosage forms). In those instances, herein, that refer to a “combination” of agents for use in a certain treatment method, simultaneous and sequential administration of the specified agents in the combination is contemplated.

In certain embodiments the compositions, combinations and methods of treatment provided herein may be employed together with other compositions, combinations and/or treatment methods known to be useful for cancer therapy (e.g., colorectal cancer therapy), including, but not limited to, surgical methods (e.g., for tumor resection), radiation therapy methods, treatment with chemotherapeutic agents, treatment with antiangiogenic agents, or treatment with tyrosine kinase inhibitors. For example, in some embodiments the methods described herein may be performed after performing surgical resection of a tumor. In other embodiments the methods described herein may be performed both before and after performing surgical resection of a tumor. Similarly, in certain embodiments the compositions, combinations and methods of treatment provided herein may be employed together with procedures used to monitor disease status/progression, such as biopsy methods and diagnostic methods (e.g., MRI methods or other imaging methods).

In some embodiments the treatment methods described herein may be employed in conjunction with performing a diagnostic test to determine if the subject has a tumor that that is likely to be responsive to therapy. For example, in some embodiments, prior to commencing treatment, a diagnostic assay is performed to determine if the subject has a microsatellite stable (MSS) cancer. Similarly, in some embodiments, prior to commencing treatment, a diagnostic assay is performed to determine if the subject has an MMR-proficient cancer. Microsatellite testing is widely used to determine the microsatellite stability / instability status and MMR status of clinical specimens. Numerous of such tests / diagnostic assays are known in the art and described in the literature, and numerous of such tests are available commercial (e.g., in kit form). Any of such tests / diagnostic assays can be used in conjunction with the present invention.

The invention is further described by the following non-limiting “Examples” and the Figures referred to therein.

### EXAMPLES

### Example 1

### Preclinical Testing

A permissive immune microenvironment and the generation of neoantigens are both needed to trigger an effective antitumoral immune response in response to treatment with immune checkpoint inhibitors (ICIs). (Luksza et al., “A neoantigen fitness model predicts tumour response to checkpoint blockade immunotherapy; ” Nature. 551, 517-520 (2017); Ribas & Wolchok, “Cancer immunotherapy using checkpoint blockade.” Science. 359, 1350-1355 (2018)). Nevertheless, most current scientific efforts are aimed at finding new immunotherapeutic combinations that modulate the immune microenvironment rather than inducing tumors to produce neoantigens. Inducing immunogenic mutations within tumor cells has the potential to convert immune “cold” tumors into a “hot” state that will benefit from immunotherapy. Using mouse tumor models, we sought to identify combinations of agents that could convert immune “cold” tumors into such a mutated “hot” state - rendering them susceptible to antitumor immune responses.

The strategy that we employed was as follows. Colon (MMR-proficient, CT26), melanoma (B16/F10) and pancreatic (Pan02) immuno-resistant mouse cancer cell lines were treated with low doses (IC₁₀ to IC₂₅) of test agents or combinations of agents chronically for a period of 8 weeks — a time frame selected as being suitable for use in a clinical setting. The immunogenicity of the chronically treated cells was examined in vitro using a syngeneic coculture assay in which the treated cells were co-cultured with immune cells. If the treatments resulted in immune-related killing at a level that was equal or superior to that exhibited by an MMR-deficient control cell line control, the genomes of the treated cells were then sequenced (whole exome sequencing 250x) to assess their genomic profiles. Finally, the immunogenicity of the treated cells was assessed in vivo by subcutaneously engrafting the treated cell lines into syngeneic mice.

We found that the combination of two approved cytotoxic drugs — temozolomide (TMZ) (known to generate missense mutations with a T to C signature and cisplatin (CDDP) (known to generate both missense and Indels mutations with a C to T signature) was particularly effective.

### Effects of TMZ and Cisplatin in Colon Cancer

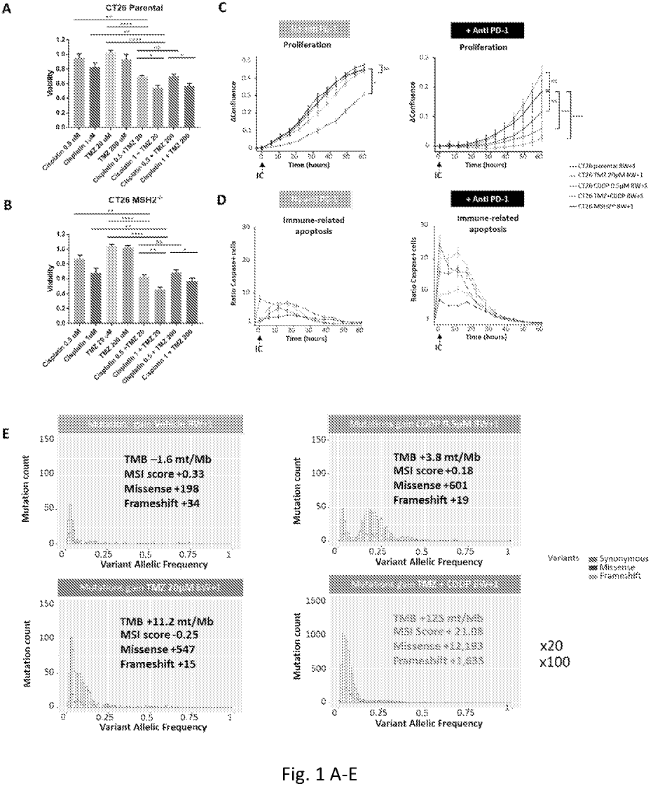

A viability/proliferation assay confirmed that the CT26 colon cancer cell line was resistant to treatment with TMZ alone but sensitive to treatment with CDDP alone. We therefore tested the effect of a low dose of TMZ (20 µM) alone, a low dose of CDDP (0.5 µM) alone, or a combination of both TMZ (20 µM) and CDDP (0.5 µM) on the proliferation/viability of these TMZ-resistant CT26 cells (FIG. 1A). We found that, while TMZ and CDDP exhibited limited cytotoxicity when used as single agents, when the cells were treated with the combination of these two agents, a greater than additive degree of cytotoxicity was observed (FIG. 1A). And when the MMR-deficient MSH2 KO cell line was treated with this combination of agents, an even greater level cytotoxicity was observed, suggesting involvement of the MMR pathway in repairing DNA damage (FIG. 1B).

To assess the immunogenic impact of chronic exposure of cancer cells to these two agents in vitro, we treated the parental CT26 colon cancer cell line with either a low dose of TMZ (20 µM) alone, a low dose of CDDP (0.5 µM) alone, or a combination of both TMZ (20 µM) and CDDP (0.5 µM) for 8 weeks and then co-cultured the treated cells with syngeneic mouse immune cells that had either had or had not been treated with a murine anti-PD-1 antibody. The results of these co-culture experiments showed that the colon cancer cells chronically treated with both TMZ and CDDP exhibited significantly higher immunogenicity in vitro as compared to those cells treated with either TMZ or CDDP as single agents (FIG. 1C, FIG. 1D). And the combination also exhibited levels of immune-related cell death similar to that observed in MSH2-/- cells treated with anti-PD-1. (“MSH2-/-” or “MSH2 KO” cells are cells in which the MSH2 protein involved in DNA repair is knocked - leading to an MMR-deficient phenotype and increased sensitivity to PD-1 blockade in mouse models. MSH2 KO cells mimic observations in MSI high patients and are used as a positive control for immunogenicity.) Of note, the TMZ single agent treatment resulted in higher levels of immune-induced apoptosis without a significant decrease in confluence as compared to the parental cell line treated by vehicle. CT26 cells treated with the TMZ/CDDP combination displayed extreme sensitivity to syngeneic immune cells exposed to anti PD-1 in co-culture experiments as compared to the untreated CT26 cells or CT26 cells treated with TMZ alone or CDDP alone. Furthermore, PD-1 sensitivity in co-cultures of CT26 cells treated with the TMZ/CDDP combination was similar to that in co-cultures of MSH2 KO cells - as reflected in both delayed growth and immune-related death.

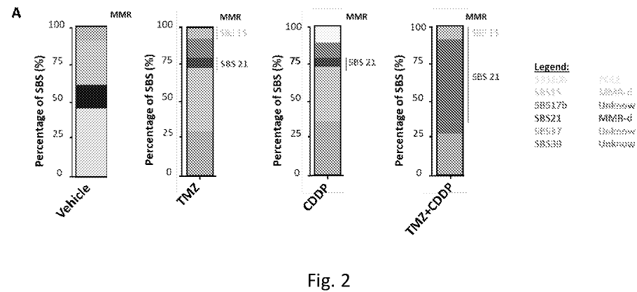

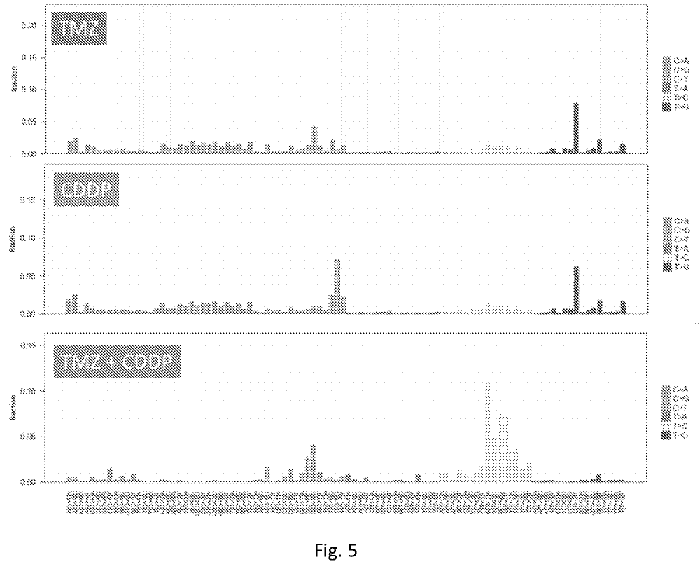

Whole exome sequencing at 250x was applied to the pre-treated cells and to cells treated with TMZ alone, CDDP alone, or the TMZ/CDDP combination (FIG. 1E). While the parental cell line had a stable tumor mutation burden (TMB) after 8 weeks and acquired few novel mutations with a low allelic frequency, the cells that were treated with the various test agents showed an increase in TMB with differential impacts depending on the treatment conditions. Treatment with CDDP caused only a moderate increase in the overall TMB, presumably because the cells that had been the most sensitive to CDDP and thus had the highest levels of mutations were killed during the chronic culture. Consequently, the CDDP-induced novel mutations were predominantly missense mutations with a higher allelic frequency. TMZ treated cells exhibited an intermediate level of increase in TMB which appeared to be mainly due to accumulation of low allelic frequency non-synonymous single nucleotide variants (“NSSNVs”), recapitulating the mutational profiles observed in long term TMZ-exposed tumors. (Campbell et al, “Comprehensive Analysis of Hypermutation in Human Cancer; ” Cell. 171, 1042-1056.e10 (2017); Touat et al., “Mechanisms and therapeutic implications of hypermutation in gliomas. ” Nature. 580, 517 523 (2020). However, the cells treated with both TMZ and CDDP displayed a massive and synergistic accumulation of novel mutations, acquiring an ultra-mutated phenotype with a TMB increasing by 125 mt/Mb. Importantly, this phenomenon involved not only increased levels of NSSNVs, but also massively increased levels of InDels/FS mutations — which were increased by 100-fold as compared to the levels in cells treated with only TMZ or only CDDP. Moreover, chronic exposure of the cells to the TMZ/CDDP combination led to an increase in the cells’; microsatellite instability score (MSI score) of 21.08 — as assessed using the MSIsensor computer program for detection of somatic microsatellite changes. (Niu et al., “MSIsensor: microsatellite instability detection using paired tumor-normal sequence data.” Bioinformatics. 30, 1015 1016 (2014)). This converted the previously microsatellite stable CT26 cells to a microsatellite instable (MSI) phenotype (MSI is a marker of mismatch repair deficiency). This was in-contrast to the effects seen when cells were treated with vehicle alone, TMZ alone, or CDDP alone - in which cases the cells remained microsatellite stable. Analysis of single base substitution (“SBS”) mutational signatures (FIG. 2) confirmed that the combination of TMZ and CDDP induced mutations in a synergistic manner with predominantly mutational profiles associated with MMR-deficiency (e.g., SBS15 and SBS21 mutational profiles; Alexandrov et al., (2020), Nature, Vol. 578, pp. 94-101), while treatment of cells with either agent alone resulted predominantly in the accumulation of the same types of mutations as occurred in the control un-treated cells. Comparison of transitions and transversions between the various treatment conditions showed that the TMZ/CDDP combination induced a unique T to C transition profile characteristic of the SBS21 mutational signature - that was not observed with either of the single agents (FIG. 5). Moreover, cells treated chronically with the TMZ/CDDP combination exhibited an Indel profile typical of of MMR-deficiency, with most of the deletions in poly T regions - defining microsatellites. Together, these results show that TMZ and CDDP synergize to induce an MMR-deficiency mutational profile, converting cancer cells to an MSI-high, ultra-mutated phenotype with an extreme accumulation of Indel/FS mutations. Importantly, functional analysis of novel copy number variants (CNVs) and non-synonymous variants in this ultra-mutated setting showed a specific loss of the short arm of the X chromosome associated with cisplatin treatment.

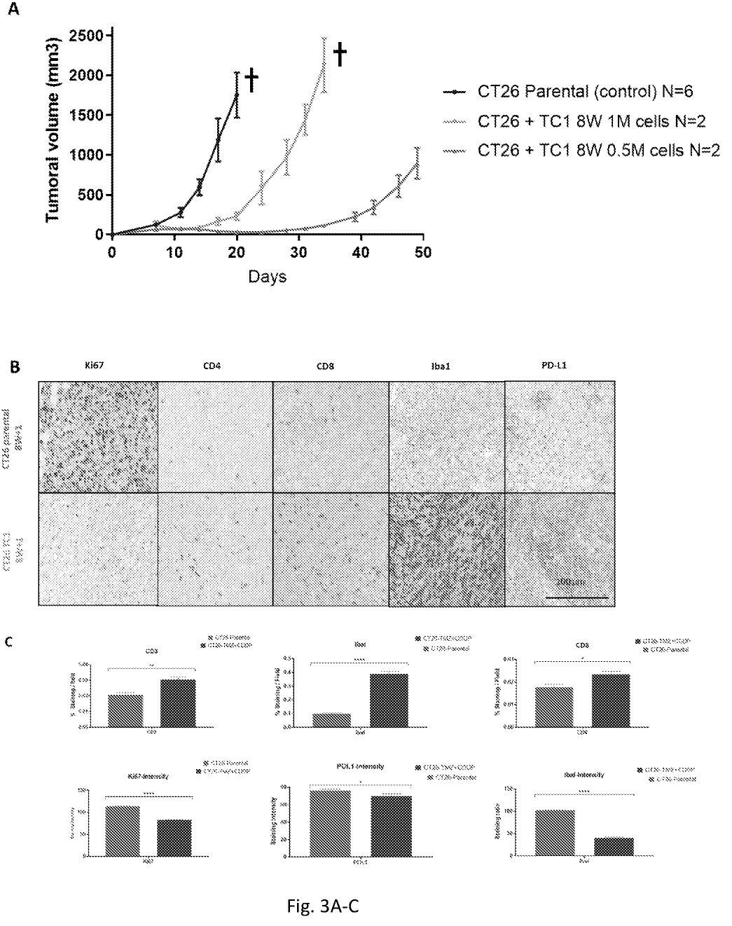

Building upon the above discoveries, we next sought to study the effects of the TMZ/CDDP combination in vivo using preclinical mouse models. Experiments were performed to assess the effects of chronic treatment with the TMZ/CDDP combination (referred to as “TC1” in some of the data figures) as compared to each single agent and/or non-treated controls. Syngeneic BALB/C mice were injected subcutaneously with CT26 cells that had been treated for 8 weeks with either vehicle alone or the TMZ/CDDP combination followed by a 1W wash out. The results are shown in FIG. 3A. While no difference in growth rate had been observed in vitro, the TMZ/CDDP treated cells exhibited delayed growth in vivo as compared to the untreated controls. Importantly, in mice inoculated with the TMZ/CDDP treated cells, we observed that 2 tumors out of 6 were rejected (following an initial period of growth). Tumor growth was also dependent on the number of cells injected. When fewer TMZ/CDDP treated cells were injected, the tumor growth was even more delayed. When 0.5 million cells were injected, tumors grew initially but after approximately 10 days began to reduce in size to the point of no-longer being palpable. The tumors then recurred at approximately 30 days. These effects were consistent with the generation of an initial immune response that was able to control the progression of the syngeneic tumors without any therapeutic intervention. Macroscopically, the tumors induced following injection of treated cells were more vascularized and softer as compared to control tumors. To confirm that the delayed tumor growth and tumor rejection was related to active immunity in the tumors, we stained tumors (generated after injection of 1 million cells) for lymphocytes, macrophages and PD-L1, and assessed proliferation by Ki67 staining (FIG. 3B). Tumors generated following injection of TMZ/CDDP treated cells displayed decreased expression of Ki67 and higher levels of CD8 T cell infiltration. While Iba1+ macrophages were uncommon in the control conditions, massive Iba1+ macrophage infiltration was observed in both experimental conditions. PD-L1 staining was patchy mainly within immune cell clusters in the control tumors but was diffuse in the experimental/treated tumors, with staining of cell membranes observed. Immune cell quantification confirmed that there was significantly higher CD3+ and CD8 T cell infiltration in the experimental/treated tumors. Higher numbers of Iba+ macrophages were observed in the experimental/treated tumors. The intensity of Ki67 staining was decreased significantly in the tumors resulting from injection of the TMZ/CDDP treated cells as compared to in the untreated parental cells. Interestingly, while the total number of PD-L1+ cells and Iba1+ cells increased in the experimental tumors, the overall intensity of PD-L1 and Iba1 expression decreased - in line with the pattern of predominant PD-L1 + cancer cell staining. Taken together, the results of these in vivo studies suggest that the growth delays seen in tumors formed from cells treated with TMZ and CDDP are immune-related, and also suggest that the treatment of cancer cells with these agents results in the induction of PD-L1 expression by the cancer cells and transforms tumors into a highly inflamed “hot” immune state rich in T-cell infiltrating lymphocytes (including CD8+ cells) and macrophages.

### Effects of TMZ and Cisplatin in Pancreatic Cancer and Melanoma

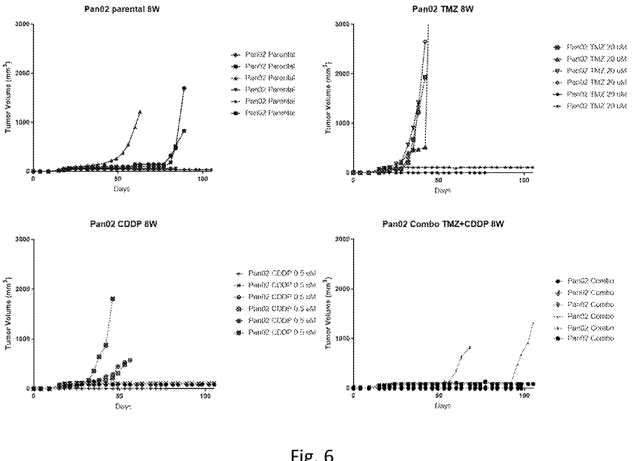

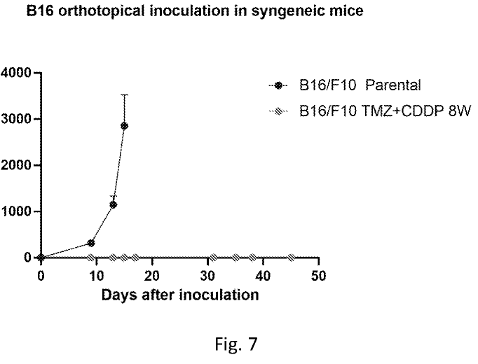

In order to assess if the above findings with colon cancer cells were applicable to other tumor types, experiments similar to those described above were performed using PD-1 resistant pancreatic cancer cells (Pan02 cell line) and PD-1 resistant melanoma cells (B16/F10 cells). Syngeneic subcutaneous engraftment of treated pancreatic cells and treated melanoma cells was performed in a C57BL/6 murine background. Results following injection of pancreatic cells and melanoma cells are presented in FIG. 6 and FIG. 7, respectively.

In the pancreatic cancer model, a typical engraftment success rate of 50 to 60% was observed. Tumors resulting from injection of pancreatic cells treated with either temozolomide alone or cisplatin alone exhibited patterns of tumor growth that were similar to each other and similar to (or even faster than) the tumor growth exhibited by tumors resulting from injection of untreated control cells. FIG. 6 Tumors resulting from injection of pancreatic cells treated with both temozolomide and cisplatin (TMZ/CDDP combination) displayed higher rejection rates and later onset of tumor progression. FIG. 6.

In the melanoma model, mice injected with melanoma cells that had been treated with both temozolomide and cisplatin (TMZ/CDDP combination) for 8 weeks exhibited rejection of all tumors, while tumors in mice injected with untreated control melanoma cells grew normally. FIG. 7.

These results suggest that the effects noted above for colorectal cancers are more generally applicable to other cancer types.

### Effects of TMZ and Cisplatin Treatment on Immune Checkpoint Blockade

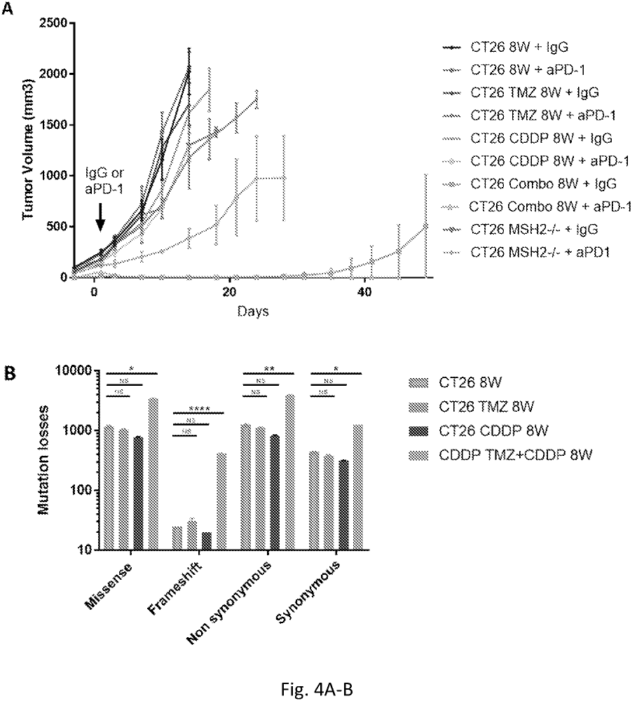

Experiments were performed to examine the effect of PD-1 blockade on the growth of tumors generated following injection of mice with tumor cells that had been pre-treated with either a vehicle control, TMZ alone, cisplatin alone, or TMZ/CDDP combination. 1 million cells from control or treatment groups (pre-treated for 8 weeks) were implanted subcutaneously into mice (n=10) and mice were then randomly assigned to either an anti PD-1 treatment group (treated with anti PD-1 at 6 mg/kg twice weekly when tumor reached 200 mm³) or an IgG isotype control group. CT26 MSH2 KO cells were used as a positive control (FIG. 4). While no effect of PD-1 blockade (as compared to isotype control) was observed for the control, temozolomide alone, or cisplatin alone treatment groups, the MSH2 KO tumors demonstrated significantly decreased growth when treated with anti PD-1 (as compared to isotype control). The results are shown in FIG. 4. For tumors generated following injection of cells pre-treated with the combination of both temozolomide and cisplatin, out of 10 implanted tumors only one was not rejected (9 out of 10 were rejected). This non-rejected tumor had been randomly assigned to the isotype control group - as opposed to the anti-PD-1 group. Because pretreatment of the cells with the combination of TMZ and cisplatin resulted in such an extreme level of tumor immuno-rejection, treatment with the immune checkpoint inhibitor was not initiated because the tumors never became established. However, given our in vitro data, including that showing that PD-L1 is highly expressed in the hypermutated TMZ+CDDP treated cells, we expected that additional in vivo studies would confirm that the addition of an immune checkpoint inhibitor would provide additional therapeutic benefit by rendering any remaining tumor cells more susceptible to anti-tumor immune responses. To confirm this, we injected mice with CT26 colon cancer cells and then challenged the mice with either vehicle alone or with a combination of both TMZ and CDDP 72 hours after engraftment (i.e. we allowed the tumor cells / tumors to become established in vivo prior to exposing them to TMZ and CDDP). In this scenario some of the tumors reached a volume of 200 mm³ - at which point we then administered either an IgG control or a anti PD-1 antibody to the mice twice a week intraperitoneally. As compared to the controls (i.e., controls treated with anti PD-1 but no TMZ/CDDP or TMZ/CDDP but no anti-PD-1), the tumors in the mice treated with both the TMZ/CDDP combination and anti-PD-1 became macroscopically highly inflamed and ulcerated - indicative of immunity being triggered by the anti PD-1 antibody specifically in the tumors treated with TMZ+CDDP.

To determine if frameshift mutations were driving the immuno-rejection of tumors triggered by the TMZ/CDDP combination, tumors isolated from mice were subjected to whole exome sequencing (250x, FIG. 4B), and immunoediting was studied by comparison with whole exome sequencing data obtained before engraftment from the isotype control groups. (In syngeneic mouse tumor models immunoediting can be assessed by quantifying mutations before implantation of tumor cells and after the cells/tumors have been allowed to grow in the mice for a period of time. Immunoediting is characterized by loss of specific mutations after tumors are grown in mice for a period of time. This loss of mutations occurs because cells expressing neoantigens are eliminated by the immune system). While no significant difference in immunoediting was observed between the vehicle control and single agent treated conditions, significantly higher levels of immunoediting were observed in the combination TMZ/CDDP treated conditions. Frameshift mutations were specifically immunoedited at a rate approximately 20-fold higher in TMZ/CDDP treated cells as compared to in cells treated with vehicle alone, TMZ alone or CDDP alone. (Minimal differences in immunoediting of non-synonymous missense mutations were observed between the various treatment conditions.) Together this data suggests that neoantigens resulting from frameshift mutations following treatment with the combination of TMZ and CDDP are highly immunogenic and are actively targeted by immune cells. In conclusion, the results presented herein demonstrate that the combination of TMZ and cisplatin: (1) increases the number of immunogenic mutations in cancer cells, 2) restores the sensitivity of cancer cells to immune checkpoint inhibition, and 3) induces immuno-rejection of tumors by transforming the immune microenvironment into a highly inflamed state.

### Example 2

### Clinical Trial of Temozolomide, Cisplatin & Nivolumab in MMR-Proficient Colorectal Cancer

Building on the preclinical studies described above, we are performing a Phase II clinical trial (ClinicalTrials.gov Identifier: NCT04457284). The goal of this clinical trial is to use temozolomide and cisplatin to induce high levels of de novo mutations, and in particular InDels, in MMR-proficient colorectal cancers, thereby mimicking the high immunogenic mutational burden associated with MSI-H / MMR-deficient colorectal cancers, and then augment this immunity with PD-1 blockade to induce an effective anti-tumor response with clinical benefit.

Each of nivolumab, TMZ and cisplatin have already been approved by the FDA for use in human patients for multiple indications and extensive details regarding the safety, pharmacology and dosing of these agents can be found in their respective Prescribing Information and Investigator Brochure - as well as in the literature. For example, a recent study in glioblastoma patients undergoing radiotherapy has shown that the combination of TMZ plus PD-1 blockade appeared feasible and well tolerated with no reported new safety concerns (Omuro et al., “OS07.3 Nivolumab in Combination with Radiotherapy with or without Temozolomide in Patients with Newly Diagnosed Glioblastoma: Updated Results From CheckMate 143.” Neuro-Oncology, 2017. 19(suppl3): p. iiil3-iiil3).

Our clinical trial is a Simon two-stage design, single arm, phase II study of patients with refractory MMR-proficient colorectal cancer (CRC), including both men and women of all races and ethnic groups. Patients have at least one tumor lesion that can be followed for RECIST 1.1 measurement. Subjects receive:
oral temozolomide (TMZ) at 150-200 mg/m2 day 1 to 5 every 4 weeks, and
cisplatin via IV infusion at 40 mg/m2 every two weeks (Q2W), and
nivolumab via IV infusion at 480 mg every four weeks (Q4W).

Treatment continues for up for 2 years maximum. The co-primary endpoints of the trial are 16-week progression free survival (PFS) rate and objective response rate (ORR) in subjects with metastatic CRC treated with TMZ, cisplatin plus nivolumab. Subjects continue treatment until progression of disease, initiation of alternative cancer therapy, unacceptable toxicity, or other reasons to discontinue treatment occur. Tumor measurements and determination of tumor responses is performed every 8 weeks according to RECIST 1.1. Subjects may continue treatment beyond radiographic progression in the absence of clinical deterioration. All subjects are followed up to 2 years for survival or until the study closes. Research studies to evaluate the effect of TMZ plus cisplatin and nivolumab are performed using archival tissue, research blood draws for ctDNA and tumor biopsies in order to measure the effect of TMZ plus cisplatin and nivolumab on the mutational profile (Missense/InDels/Frameshift), TMB and the anti-tumor immune response. The primary end points of this Phase II study are to determine the 16-week PFS and the response rate (RR, complete response plus partial response according to RECIST 1.1) in patients with CRC treated with TMZ, cisplatin and nivolumab.

We claim:

1. A method of treating an MMR-proficient and/or microsatellite-stable colorectal cancer in a subject in need thereof, the method comprising: administering to a subject with an MMR-proficient and/or microsatellite-stable colorectal cancer an effective amount of: (a) temozolomide, (b) cisplatin, and (c) nivolumab, thereby treating the MMR-proficient and/or microsatellite stable colorectal cancer in the subject.

2. The method of claim 1, further comprising performing a test to determine if the subject has a MMR-proficient and/or microsatellite-stable colorectal cancer prior to admninistering the temozolomide, cisplatin and nivolumab.

3. The method of claim 1, wherein the cancer is immune checkpoint inhibitor resistant.

4. The method of claim 1, wherein the cancer was previously treated with, and exhibited resistance to, one or more immune checkpoint inhibitors.

5. The method of claim 1, wherein the cancer is nivolumab resistant.

6. The method of claim 1, wherein the cancer was previously treated with, and exhibited resistance to, nivolumab.

7. The method of claim 1, wherein the nivolumab is administered by IV infusion.

8. The method of claim 1, wherein the temozolomide is administered orally.

9. The method of claim 1, wherein the temozolomide is administered by IV infusion.

10. The method of claim 1, wherein the cisplatin is administered by IV infusion.

11. The method of claim 1, wherein the nivolumab is administered to the subject by IV infusion about at about 480 mg about every 4 weeks (480 mg Q4W).

12. The method of claim 1, wherein the temozolomoide is administered to the subject orally at about 50-200 mg/m2, day 1-5, about every 4 weeks (Q4W).

13. The method of claim 1, wherein the cisplatin is administered to the subject by IV infusion at about 40 mg/m2 about every 2 weeks (40 mg/m2 Q2W).

14. A method of treating an MMR-proficient and/or microsatellite-stable cancer in a subject in need thereof, the method comprising: administering to a subject with MMR-proficient and/or microsatellite-stable cancer an effective amount of: (a) an imidazotetrazine chemotherapeutic agent, and (b) a platinum-containing chemotherapeutic agent, thereby treating the MMR-proficient and/or microsatellite stable cancer in the subject.

15. The method of claim 14, further comprising administering to the subject an effective amount of an immune checkpoint inhibitor.

16. The method of claim 14 or claim 15, further comprising performing a test to determine if the subject has a MMR-proficient and/or microsatellite-stable cancer prior to administering the imidazotetrazine chemotherapeutic agent, platinum-containing chemotherapeutic agent and/or immune checkpoint inhibitor to the subject.

17. The method of any of claims 14-16, wherein the cancer is colorectal cancer, pancreatic cancer, or melanoma.

18. The method of any of claims 14-16, wherein the cancer is colorectal cancer.

19. The method of claims 14-18, wherein the cancer is immune checkpoint inhibitor resistant.

20. The method of any of claims 14-18, wherein the cancer was previously treated with, and exhibited resistance to, one or more immune checkpoint inhibitors.

21. The method of any of claims 14-20, wherein the imidazotetrazine chemotherapeutic agent is selected from the group consisting of TMZ and dacarbazine.

22. The method of any of claims 14-20, wherein the imidazotetrazine chemotherapeutic agent is TMZ.

23. The method of any of claims 14-22, wherein the platinum-containing chemotherapeutic agent is selected from the group consisting of cisplatin, carboplatin and oxaliplatin.

24. The method of any of claims 14-22, wherein the platinum-containing chemotherapeutic agent is cisplatin.

25. The method of any of claims 15-24, wherein the immune checkpoint inhibitor is a PD-1, PD-L1, PD-L2 or CTLA-4 inhibitor.

26. The method of any of claims 15-24, where the immune checkpoint inhibitor is selected from the group consisting of: nivolumab, pembrolizumab, tremelimumab, ipilimumab. cemiplimab, MPDL3280A, AMP-224, AMP-514 and PDR001, atezolizumab, Avelumab, Durvalumab, BMS-936559, CK-301 , tislelizumab, toripalimab, envafolimab, HLX10, and HLX20.

27. The method of any of claims 15-24, where the immune checkpoint inhibitor is nivolumab.

28. The method of any of any of claims 15-27, wherein the immune checkpoint inhibitor is is administered by IV infusion.

29. The method of any of claims 14-27, wherein the temozolomeide is administered orally.

30. The method of any of claims 14-27wherein the temozolomeide is administered by IV infusion.

31. The method of any claims 14-27, wherein platinum-containing chemotherapeutic agent is administered by IV infusion.

32. The method of any of claims 15-27, wherein the immune checkpoint inhibitor is nivolumab, and wherein the nivolumab is administered to the subject by IV infusion about at about 480 mg about every 4 weeks (480 mg Q4W).

33. The method of any of claims 14-27, wherein the temozolomoide is administered to the subject orally or by IV infusion at about 50-200 mg/m2, day 1-5, about every 4 weeks (Q4W).

34. The method of any of claims 14-27, wherein the temozolomoide is administered to the subject by IV infusion at about 75 mg/m2 daily.

35. The method of any of claims 14-27, wherein the platinum-containing chemotherapeutic agent is cisplatin, and wherein the cisplatin is administered to the subject by IV infusion at about 30 mg/m2 weekly, or at about 40 mg/m2 about every 2 weeks (40 mg/m2 Q2W), or at about 60 to 100 mg/m2 every 3 to 4 weeks.

36. The method of any of claims 14-27, wherein the platinum-containing chemotherapeutic agent is cisplatin, and wherein the cisplatin is administered to the subject by IV infusion at about 75 mg/m2 daily.

37. The method of any of the preceding claims wherein the method enhances the immunogenicity of MMR-proficient and/or microsatellite stable (MSS) cancer cells in the subject.

38. The method of any of the preceding claims wherein the method enhances the sensitivity of MMR-proficient and/or microsatellite stable (MSS) cancer cells in the subject to immune checkpoint blockade.

39. The method of any of the preceding claims wherein the method induces an MMR-deficient mutational signature in MMR-proficient and/or microsatellite stable (MSS) cancer cells in the subject.

40. The method of any of the preceding claims wherein the method increases the frequency of both missense and InDel mutations in MMR-proficient and/or microsatellite stable (MSS) cancer cells in the subject.

41. A method of enhancing the immunogenicity of MMR-proficient and/or microsatellite stable (MSS) cancer cells, the method comprising contacting the cancer cells with an effective amount of an imidazotetrazine chemotherapeutic agent and a platinum-containing chemotherapeutic agent.

42. The method of claim 41, wherein the imidazotetrazine chemotherapeutic agent is selected from the group consisting of TMZ and dacarbazine.

43. The method of claim 41, wherein the imidazotetrazine chemotherapeutic agent is TMZ.

44. The method of any of claims 41-43, wherein the platinum-containing chemotherapeutic agent is selected from the group consisting of cisplatin, carboplatin and oxaliplatin.

45. The method of any of claims 41-43, wherein the platinum-containing chemotherapeutic agent is cisplatin.

46. The method of any of claims 41-45, wherein the cancer cells are colorectal cancer cells.

47. The method any of claims 41-45, wherein the cancer cells are pancreatic cancer cells.

48. The method of any of claims 41-45, wherein the cancer cells are melanoma cells.

49. The method of any of claims 41-48 wherein the cancer cells are in a subject and wherein the method comprises administering the temozolomide and the platinum-containing chemotherapeutic agent to the subject.

50. A method of enhancing the sensitivity of MMR-proficient and/or microsatellite stable (MSS) cancer cells to immune checkpoint blockade, the method comprising contacting the cancer cells with an effective amount of an imidazotetrazine chemotherapeutic agent and a platinum-containing chemotherapeutic agent.

51. The method of claim 50, wherein the imidazotetrazine chemotherapeutic agent is selected from the group consisting of TMZ and dacarbazine.

52. The method of claim 50, wherein the imidazotetrazine chemotherapeutic agent is TMZ.

53. The method of any of claims 50-52, wherein the platinum-containing chemotherapeutic agent is selected from the group consisting of cisplatin, carboplatin and oxaliplatin.

54. The method of any of claims 50-52, wherein the platinum-containing chemotherapeutic agent is cisplatin.

55. The method of any of claims 50-54, wherein the cancer cells are colorectal cancer cells.

56. The method of any of claims 50-54, wherein the cancer cells are pancreatic cancer cells.

57. The method of any of claims 50-54, wherein the cancer cells are melanoma cells.

58. The method of any of claims 50-57, wherein the cancer cells are in a subject and wherein the method comprises administering the temozolomide and the platinum-containing chemotherapeutic agent to the subject.

59. A method of inducing an MMR-deficient mutational signature in MMR-proficient and/or microsatellite stable (MSS) cancer cells, the method comprising contacting the cancer cells with an effective amount of temozolomide and a platinum-containing chemotherapeutic agent.

60. The method of claim 59, wherein the imidazotetrazine chemotherapeutic agent is selected from the group consisting of TMZ and dacarbazine.

61. The method of claim 59, wherein the imidazotetrazine chemotherapeutic agent is TMZ.

62. The method of any of claims 59-61, wherein the platinum-containing chemotherapeutic agent is selected from the group consisting of cisplatin, carboplatin and oxaliplatin.

63. The method of any of claims 59-61, wherein the platinum-containing chemotherapeutic agent is cisplatin.

64. The method of any of claims 59-63, wherein the cancer cells are colorectal cancer cells.

65. The method of any of claims 59-63, wherein the cancer cells are pancreatic cancer cells.

66. The method of any of claims 59-63, wherein the cancer cells are melanoma cells.

67. The method of any of claims 59-66, wherein the cancer cells are in a subject and wherein the method comprises administering the temozolomide and the platinum-containing chemotherapeutic agent to the subject.

68. A method of increasing the frequency of both missense and InDel mutations in MMR-proficient and/or microsatellite stable (MSS) cancer cells, the method comprising contacting the cancer cells with an effective amount of an imidazotetrazine chemotherapeutic agent and a platinum-containing chemotherapeutic agent.

69. The method of claim 68, wherein the imidazotetrazine chemotherapeutic agent is selected from the group consisting of TMZ and dacarbazine.

70. The method of claim 68, wherein the imidazotetrazine chemotherapeutic agent is TMZ.

71. The method of any of claims 68-70, wherein the platinum-containing chemotherapeutic agent is selected from the group consisting of cisplatin, carboplatin and oxaliplatin.

72. The method of any of claims 68-70, wherein the platinum-containing chemotherapeutic agent is cisplatin.

73. The method of any of claims 68-72, wherein the cancer cells are colorectal cancer cells.

74. The method of any of claims 68-72, wherein the cancer cells are pancreatic cancer cells.

75. The method of any of claims 68-72, wherein the cancer cells are melanoma cells.

76. The method of any of claims 68-75, wherein the cancer cells are in a subject and wherein the method comprises administering the imidazotetrazine chemotherapeutic agent and the platinum-containing chemotherapeutic agent to the subject.

77. A pharmaceutical composition comprising: (a) an imidazotetrazine chemotherapeutic agent and (b) a platinum-containing chemotherapeutic agent.

78. A pharmaceutical composition comprising: (a) TMZ and (b) cisplatin.

79. A pharmaceutical composition comprising: (a) a platinum-containing chemotherapeutic agent, and (b) an immune checkpoint inhibitor.

80. A pharmaceutical composition comprising: (a) cisplatin, and (b) nivolumab.

81. A pharmaceutical composition comprising: (a) an imidazotetrazine chemotherapeutic agent, (b) a platinum-containing chemotherapeutic agent, and (c) an immune checkpoint inhibitor.

82. A pharmaceutical composition comprising: (a) temozolomide, (b) cisplatin, and (c) nivolumab.

83. A pharmaceutical composition according to claim 77-82, for use in treatment of a MMR-proficient and/or microsatellite stable cancer in a subject in need thereof.

84. A pharmaceutical composition according to claim 77-82, for use in treatment of a MMR-proficient and/or microsatellite stable colorectal cancer in a subject in need thereof.

85. A pharmaceutical composition according to claim 77-82, for use in treatment of a MMR-proficient and/or microsatellite stable pancreatic cancer in a subject in need thereof.

86. A pharmaceutical composition according to claim 77-82, for use in treatment of a MMR-proficient and/or microsatellite stable melanoma in a subject in need thereof.

87. A combination comprising: (a) an imidazotetrazine chemotherapeutic agent and (b) a platinum-containing chemotherapeutic agent, for use in treatment of a MMR-proficient and/or microsatellite stablecancer in a subject in need thereof.

88. A combination comprising: (a) TMZ and (b) cisplatin, for use in treatment of a MMR-proficient and/or microsatellite stablecancer in a subject in need thereof.

89. A combination comprising: (a) a platinum-containing chemotherapeutic agent, and (b) an immune checkpoint inhibitor, for use in treatment of a MMR-proficient and/or microsatellite stablecancer in a subject in need thereof.

90. A combination comprising: (a) cisplatin, and (b) nivolumab, for use in treatment of a MMR-proficient and/or microsatellite stablecancer in a subject in need thereof.

91. A combination comprising: (a) an imidazotetrazine chemotherapeutic agent, (b) a platinum-containing chemotherapeutic agent, and (c) an immune checkpoint inhibitor, for use in treatment of a MMR-proficient and/or microsatellite stablecancer in a subject in need thereof.

92. A combination comprising: (a) temozolomide, (b) cisplatin, and (c) nivolumab, for use in treatment of a MMR-proficient and/or microsatellite stablecancer in a subject in need thereof.

93. The combination for use of any of claims 87-92, wherein the MMR-proficient and/or microsatellite stable cancer is a colorectal cancer.

94. The combination for use of any of claims 87-92, wherein the MMR-proficient and/or microsatellite stable cancer is a pancreatic cancer.

95. The combination for use of any of claims 87-92, wherein the MMR-proficient and/or microsatellite stable cancer is a melanoma.
